(19) United States
(12) Patent Application Publication
(10) Pub. No.: US 2005/0058683 A1
(43) Pub. Date: Mar. 17, 2005

(54) Absorbent articles with antimicrobial zones on coverstock

(76) Inventors: Ruth L. Levy, Collegeville, PA (US); Lanying Wu, Paoli, PA (US)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(21) Appl. No.: 10/661,020

(22) Filed: Sep. 12, 2003

(51) Int. Cl. A61K 31/785

(52) U.S. Cl. 424/423; 424/78.35

(57) ABSTRACT

The disclosure describes compositions, such as absorbent article topsheets or undersheets, that on its bodyside surfaces comprises an anionic surfactant-bound polymeric biguanide or an anionic surface-bound polymeric biguanide that can dissociate from the article so as to attack bacteria on the wearer's skin.

### BACKGROUND OF THE INVENTION

1. Field of Invention

This invention relates to absorbent articles such as disposable diapers, disposable incontinence pads and disposable feminine hygiene pads that, on their topsheet surface, comprise an anti-microbial agent.

2. Description of Related Art

A desirable property goal of absorbent materials such as disposable diapers, incontinence pads or feminine hygiene pads, is that the urine they absorb does not become a haven or breeding medium for bacteria. The bacteria are undesirable because they catalyze the breakdown of substances in the urine, creating products that cause a strong odor and skin irritation.

U.S. Pat. No. 4,655,756, describes an absorbent article that has the antibacterial compound, polyhexamethylene biguanide (PHMB), in its nonwoven core material. U.S. Pat. No. 5,993,840 similarly discloses the use of PHMB in the core of a diaper. Additionally, however, it provides for a PHMB-binding anionic polymer in the core in order to retain the PHMB there.

The aforementioned patents disclose inventions designed to attack bacteria in urine absorbed by the article's core material. The present invention can, prior to or after urination, attack bacteria that resides either on the wearer's skin or on the article topsheet itself.

### BRIEF SUMMARY OF THE INVENTION

In a general aspect, the invention is a composition that comprises a material with an anionic surfactant on its surface and a polymeric biguanide noncovalently bonded to the anionic surfactant.

In a related aspect, the invention is a composition that comprises a material whose surface is anionic (is negatively charged) and that further comprise polymeric biguanide noncovalently bonded to that anionic surface. Examples of materials that have such an anionic surface include but are not limited to cotton, pulp, and rayon.

For both of the above aspects of the invention, any anionic surfactant bound to the surface is considered an entity that is different from the surface itself.

The anionic surface or surfactant to which the polymeric biguanide is bound serves as a reservoir from which the polymeric biguanide is released upon contact of the material with bacteria-populated skin. Sufficient polymeric biguanide is released so as to bind to the bacteria and either kill them or prevent their further growth.

In preferred embodiments, the polymeric biguanide is limited to a zone on the surface. If used, the anionic surfactant can be similarly limited. In many preferred embodiments, such a zone occupies between 10% and 50% of the surface area.

Examples of compositions of interest include, but are not limited to those comprising fibers, those that are liquid pervious nonwoven webs (preferably with a weight in the range, 10 to 30 grams per square meter) and those that are liquid pervious apertured films (preferably with a weight in the range 15 to 40 grams per square meter.). Fibers of interest for such compositions include, but are not limited to staple fibers and continuous fibers. Fibers can be part of a carded web (especially staple fibers), spunbond (especially continuous fibers) be thermally or ultrasonically bonded, adhesively bonded, bonded by hydroentangling, or combined in other ways known in the art.

A related invention is an absorbent article that comprises, as a topsheet, a composition of the invention. As it is the surface intended to contact the skin of the person wearing the article, the bodyside surface of the topsheet will comprise the polymeric biguanide and, if used, the anionic surfactant. Some examples of such absorbent articles are a disposable incontinence pad, a disposable diaper, and a disposable feminine hygiene pad. The article optionally further comprises, in its core, a polymeric biguanide, or both a polymeric biguanide and an anionic surfactant.

In a variation of the absorbent article, the polymeric biguanide is located on the topside surface of a layer that is just under the article's topsheet. (The topside surface is therefore the one in contact with the topsheet.) Typically such an article comprises the top sheet, an intermediate layer (such as an acquisition layer), an absorbent layer and a backsheet. {Paralleling the compositions of the invention, either the topside surface of the intermediate layer is anionic or an anionic surfactant is bound to the topside surface. Accordingly, the polymeric biguanide is bound to that anionic topside surface or to that anionic surfactant. Although the topside surface of the intermediate layer may not directly contact the skin of the wearer, the fact that the topsheet can be very thin and liquid pervious means that that perspiration from the wearer can mediate interaction between the polymeric biguanide and bacteria on the wearer's skin.

In particular embodiments, the composition of the invention is produced by a process that comprises (1) pre-treating the composition's surface with the anionic surfactant, and (2) then applying the polymeric biguanide to the surface so that it binds to the anionic surfactant. If the surface is anionic, the first step can be omitted and the polymeric biguanide is applied so that it binds directly to the surface. The aforementioned processes are themselves aspects of the invention and are of particular interest here where the composition is the topsheet or intermediate layer for a disposable article.

One set of options for applying a polymeric biguanide or anionic surfactant is to use a kiss-on or brush roller. Another is to use a spray or foam comprising the polymeric biguanide. The anionic surfactant may be added as a melt-additive, especially when the composition is a fiber. Additional options for applying the biguanide or surfactant are known in the art.

### BRIEF DESCRIPTION OF THE DRAWINGS

FIG. 1A is an exploded perspective view of a disposable diaper.

FIG. 1B is a planar view of the bodyside surface of the topsheet in FIG. 1A.

### DETAILED DESCRIPTION OF THE INVENTION

Polymeric Biguanides

One class of preferred polymeric biguanides are linear polymeric biguanides, described in U.S. Pat. Nos. 4,655,756 and 5,993,480, in which the recurring unit is of the formula, or a salt thereof: 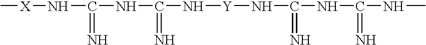
wherein X and Y are bridging groups that may be the same or different, such that the number of N to N carbon atoms within X plus the number of N to N carbon atoms within Y is in the range 9 to 17, where the number of N to N carbon atoms within a bridging group is the number of backbone carbon atoms that, in that group, separate the N atoms adjacent to that group. (For example, for hexamethylene, the distance is 6) or the salt thereof with an acid. X any Y preferably comprise polymethylene chains.

X and Y are, for example, polymethylene chains. Those chains optionally incorporate heteroatoms such as oxygen, sulfur or nitrogen. An example of a chain with such incorporation is ethylene oxyethylene. Furthermore the chains optionally incorporate saturated or unsaturated cyclic nuclei. In those cases, the N to N distance refers to the shortest of the two possible N to N routes along the cyclic moiety.

A class of preferred polymeric biguanides are polyhexamethylene biguanides of the formula:
Zₙ
where Z is 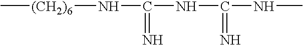
and n is from 2 to 40.
PHMB

PHMB is the most preferred polymeric biguanide. It is highly preferred because, at very low concentrations, it has a broad spectrum of activity against bacteria, fungi and yeasts, particularly those associated with the human body. It is also harmless to the macrobiotic system and is not a skin sensitizer. Therefore, it will not give rise to problems such as skin irritation or rashes when applied to products that directly contact the skin.

At relatively low concentrations, PHMB is bacteriostatic. At higher concentrations, it is rapidly bacteriocidal. PHMB achieves its negative effect on a bacterium by initially binding to a receptive site on the microbe's surface, and then proceeding to disrupt its cytoplasmic membrane.

Anionic Surfactant

The topsheet of the non-woven material is treated with an anionic surfactant that will not be washed away during repeated insults. This surfactant should, in turn, bind the polymeric biguanide sufficiently strongly to prevent most of it from being washed away during normal insults to the absorbent article. On the other hand, the surfactant must bind the polymeric biguanide sufficiently weakly to allow some of it to migrate to and kill microbes.

The solubility of the anionic surfactant in urine is preferably not greater than 2%, more preferably not greater than 1%, even more preferably not greater than 0.5% and especially not greater than 0.1% by weight. At temperature in the range 20° C. to 37° C.

Anionic polymers useful for binding polymeric biguanides are disclosed and discussed in U.S. Pat. No. 5,993,840. The anionic polymer can be obtained by polymerization or copolymerization of appropriate monomers. The anionic group may be a phosphonic, phosphoric, sulphonic, or carboxylic group. Carboxylic groups are preferred.

Possible anionic monomers include, but are not limited to, vinylphosponic acid, styrene-phosphonic acid, 2-acrylamidopropanephosphonic acid, ethylidene-1,1-diphosphonic acid, hydroxyethylacrylate monophosphate, styrene sulphonic acid, 2-acrylamido-2-methylpropanesulphonic acid, sulphoethyl methacrylate, vinylsulphonic acid, methallyl sulphonic acid, propene sulphonic acid and, more preferably, methacrylic and, even more preferably, acrylic acid.

Preferred anionic polymers are:

polyacrylic acid and copolymers of acrylic acid with one or more non-ionic monomers;

poly(maleic acid) and copolymers thereof with one or more non-ionic monomers;

alginic acid;

graft polymers of acrylic acid unto starch; and

graft polymers of acrylic acid unto carboxymethylcellulose.

Other anionic polymers that can be used are those derived from carboxymethylcellulose, partially oxidized cellulose, sulphoethylcellulose, or phosphorylated cellulose.

Useful non-ionic monomers include, but are not limited to, those of the formula

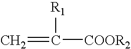

where R₁ is hydrogen or C₁-C₄ alkyl (an alkyl group of 1 to 4 carbon atoms);

where R₂ is alkyl (preferably C₁-C₂₀ alkyl, more preferably C₁-C₆ alkyl), aryl (preferably phenyl) or cycloalkyl (preferably cyclohexyl) . For each of said R₂ moieties, R₂ is optionally substituted.

Preferred nonionic monomers are methyl(meth)acrylate, butyl(meth)acrylate, ethyl(meth)acrylate, 2-ethylhexyl(meth)acrylate, 2-hydroxyethyl(meth)acrylate and acetoacetoxyethyl(meth)acrylate.

Preferred anionic polymers are polyacrylic acid and copolymers of acrylic acid that have one or more non-ionic monomers, poly(maleic acid) and copolymers of maleic acid and at least one nonionic monomer, alginic acid, graft polymers of acrylic acid onto starch and carboxymethyl cellulose.

Polyacrylic acid will preferably be in the molecular weight range 1000 to 5,000,000.

The polymers are, optionally, crosslinked.

Super absorbent polymers, such as those based on polyacrylates and related polymers are highly preferred.

The surfactant can, for example, be applied by spraying. Alternatively, it can be applied by dipping the topsheet in a bath of the surfactant.

Interaction of Polymeric Biguanides with the Surfactant or Anionic Surface

The polymeric biguanide, especially if it is a mild cationic compound such as PHMB, will interact with the anionic surfactant or surface to form cationic-anionic pairs. The surfactant tethers the polymeric biguanide to the surface, hindering but not completely preventing migration of biguanide away from the topsheet. An anionic surface binds the biguanide directly, similarly hindering its migration. As long as a substantial amount of the polymeric biguanide is present on the topsheet, it will provide a reservoir for polymeric biguanide that can dissociate from the topsheet and bind to the skin bacteria.

The polymeric biguanide is preferably applied through a “kiss-on” roller or brush roller. Therefore, ion pairs will be formed only at the areas that have direct contact with the roller. Such areas of the absorbent material preferably include the topsheet areas that have direct contact with the skin. The amount of polymeric biguanide applied is preferably between 8 and 19 mg/SM (mg/square meter) . The hydrochloride salt of the polymeric biguanide can be used.

Topsheet

The topsheet (coverstock) is preferably liquid pervious, soft and non-irritating to the wearer's skin. A suitable topsheet may be manufactured from a wide range of woven or nonwoven materials. Such materials include, but are not limited to, polymeric material such as apertured formed thermoplastic films, apertured plastic films, and hydro-formed thermoplastic films. If nonwoven, the web may, for example, be one that was spun-bonded, carded, wet-laid, melt-blown, hydro-entangled, or made by a combination of two or more such techniques.

The invention will be illustrated in more detail with reference to the following Example, but it should be understood that the present invention is not deemed to be limited thereto.

### EXAMPLE

### Example 1

### Method for Producing an Absorbent Material with the Anti-Microbial PHMB on the Surface

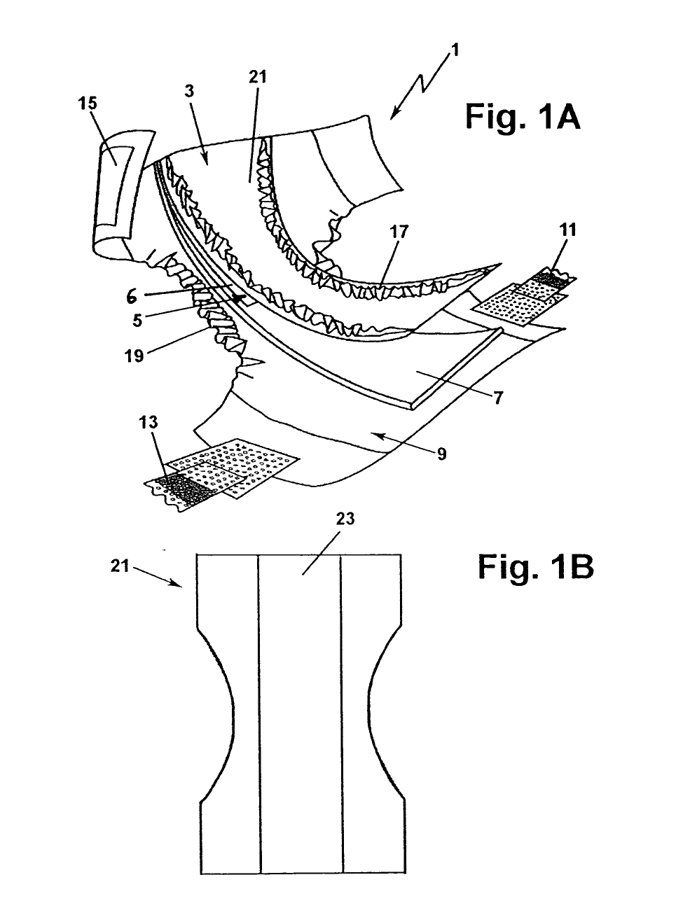

FIG. 1A illustrates a disposable diaper 1 in which a topsheet 3 is used. The topsheet 3, acquisition layer (intermediate layer) 5, absorbent layer (core) 7 and back sheet 9 form successive layers. Also evident are attachment tapes 11, 13, a tape landing area 15, a standing leg cuff 17, and an elastic gatherer 19. The surface 21 of the topsheet is the bodyside surface; i.e., the side that will come in contact with the skin of a wearer's body. The surface 6 of the acquisition layer is the topside surface of that layer.

The bodyside surface 21 of the topsheet 3 is shown in FIG. 1B. Prior to assembly of the diaper, a zone 23 of that surface is sprayed with the permanent anionic surfactant, crosslinked polyacrylate, dissolved in water at a concentration of 20% by weight in water so that the zone 23 has a surfactant concentration of about 8-200 g/square meter. The topsheet is dried using a hot air blower.

PHMB, at a concentration of 0.1 to 1.0% wt/wt in water, is applied to the surfactant-coated zone using a kiss-on or brush roller so as to achieve a concentration of 8-19 mg/SM in water in the zone. The topsheet is then dried in a vacuum.

While the invention has been described in detail and with reference to specific examples thereof, it will be apparent to one skilled in the art that various changes and modifications can be made therein without departing from the spirit and scope thereof.

1. A composition comprising:
a) a material, said material comprising a surface;
b) an anionic surfactant on said surface; and
c) a polymeric biguanide noncovalently bonded to said anionic surfactant.

2. The composition of claim 1, wherein the polymeric biguanide is a recurring unit of the following formula, or a salt thereof: 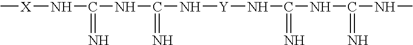
wherein X and Y are bridging groups that may be the same or different, such that the number of N to N carbon atoms within X plus the number of N to N carbon atoms within Y is in the range 9 to 17, where the number of N to N carbon atoms within a bridging group is the number of backbone carbon atoms that, in that group, separate the n atoms adjacent to that group.

3. A composition of claim 2 wherein the polymeric biguanide is polyhexamethylene biguanide (PHMB).

4. The composition of claim 1 wherein the concentration of the polymeric biguanide is in the range 5 to 50 mg per square meter of the surface.

5. The composition of claim 4 wherein the concentration of the polymeric biguanide is in the range 8 to 19 mg per square meter of the surface.

6. The composition of claim 1 wherein the polymeric biguanide is limited to a zone of said surface.

7. The composition of claim 6 wherein the concentration of the polymeric biguanide in the zone is in the range 5 to 50 mg per square meter of the zone.

8. The composition of claim 7 wherein the concentration of the polymeric biguanide in the zone is in the range 8 to 19 mg per square meter of the zone.

9. The composition of claim 1, wherein said composition is produced by a process comprising the steps of:
(1) pre-treating the surface of the composition with the anionic surfactant, and then
(2) applying the polymeric biguanide to the surface.

10. The composition of claim 9 wherein the polymeric biguanide is applied to the surface using a kiss-on or brush roller.

11. The composition of claim 10, wherein the polymeric biguanide is a recurring unit of the following formula, or a salt thereof: 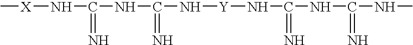
wherein X and Y are bridging groups that may be the same or different, such that the number of N to N carbon atoms within X plus the number of N to N carbon atoms within Y is in the range 9 to 17, where the number of N to N carbon atoms within a bridging group is the number of backbone carbon atoms that, in that group, separate the N atoms adjacent to that group.

12. A composition of claim 11 wherein the polymeric biguanide is PHMB.

13. The composition of claim 9 wherein the polymeric biguanide is applied to the surface using a spray or foam comprising the polymeric biguanide.

14. The composition of claim 1 wherein the material is a liquid-pervious nonwoven web.

15. The composition of claim 14 wherein the web has a weight in the range 10 to 30 gram per square meter.

16. The composition of claim 1 wherein the material is a liquid-pervious apertured film.

17. The composition of claim 16 wherein the material has a weight in the range 15 to 40 grams per square meter.

18. The composition of claim 1 wherein the material comprises fibers.

19. The composition of claim 18 wherein the fibers are selected from the group consisting of staple fibers and continuous fibers.

20. The composition of claim 18 wherein the fibers are part of a carded web.

21. The composition of claim 18 wherein the fibers have been spunbond, thermally bonded, ultrasonically bonded, adhesively bonded, or combined by hydroentangling.

22. The composition of claim 18 wherein the anionic surfactant was added as a melt additive to the fibers.

23. The composition of claim 1 wherein the composition is a topsheet for use in an absorbent article.

24. The composition of claim 23 wherein the absorbent article is selected from the group consisting of a disposable diaper, a disposable incontinence pad and a disposable feminine hygiene pad.

25. A composition comprising:
a) a material, said material comprising an anionic surface; and
b) a polymeric biguanide noncovalently bonded to said anionic surface.

26. The composition of claim 25, wherein the polymeric biguanide is a recurring unit of the following formula, or a salt thereof: 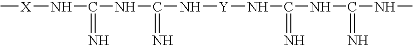
wherein X and Y are bridging groups that may be the same or different, such that the number of N to N carbon atoms within X plus the number of N to N carbon atoms within Y is in the range 9 to 17, where the number of N to N carbon atoms within a bridging group is the number of backbone carbon atoms that, in that group, separate the N atoms adjacent to that group.

27. A composition of claim 26 wherein the polymeric biguanide is PHMB.

28. The composition of claim 25 wherein the concentration of the polymeric biguanide is in the range 5 to 50 mg per square meter of the surface.

29. The composition of claim 28 wherein the concentration of the polymeric biguanide is in the range 8 to 19 mg per square meter of the surface.

30. The composition of claim 25 wherein the polymeric biguanide is limited to a zone of said surface.

31. The composition of claim 30 wherein the concentration of the polymeric biguanide in the zone is in the range 5 to 50 mg per square meter of the zone.

32. The composition of claim 31 wherein the concentration of the polymeric biguanide in the zone is in the range 8 to 19 mg per square meter of the zone.

33. The composition of claim 25, wherein said composition is produced by a process comprising the steps of:
(2) pre-treating the surface of the composition with the anionic surfactant, and then
(2) applying the polymeric biguanide to the surface.

34. The composition of claim 33 wherein the polymeric biguanide is applied to the surface using a kiss-on or brush roller.

35. The composition of claim 34, wherein the polymeric biguanide is a recurring unit of the following formula, or a salt thereof: 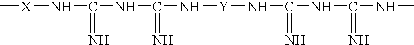
wherein X and Y are bridging groups that may be the same or different, such that the number of N to N carbon atoms within X plus the number of N to N carbon atoms within Y is in the range 9 to 17, where the number of N to N carbon atoms within a bridging group is the number of backbone carbon atoms that, in that group, separate the N atoms adjacent to that group.

36. A composition of claim 35 wherein the polymeric biguanide is PHMB.

37. The composition of claim 33 wherein the polymeric biguanide is applied using a spray or foam comprising the polymeric biguanide.

38. The composition of claim 25 wherein the material is a liquid-pervious nonwoven web.

39. The composition of claim 38 wherein the web has a weight in the range 10 to 30 gram per square.

40. The composition of claim 25 wherein the material is a liquid-pervious apertured film.

41. The composition of claim 40 wherein the material has a weight in the range 15 to 40 grams per square meter.

42. The composition of claim 25 wherein the material comprises fibers.

43. The composition of claim 42 wherein the fibers are selected from the group consisting of staple fibers and continuous fibers.

44. The composition of claim 42 wherein the fibers are part of a carded web.

45. The composition of claim 42 wherein the fibers have been spunbond, thermally bonded, ultrasonically bonded, adhesively bonded, or bonded by hydroentangling.

46. The composition of claim 25 wherein the composition is a topsheet for use in an absorbent article.

47. The composition of claim 46 wherein the absorbent article is selected from the group consisting of a disposable diaper, a disposable incontinence pad and a disposable feminine hygiene pad.

48. An absorbent article that comprises, as a topsheet, a composition of claim 1.

49. An absorbent article of claim 48, said article selected from the group consisting of a disposable diaper, a disposable incontinence pad and a disposable feminine hygiene pad.

50. An absorbent article that comprises, as a topsheet, a composition of claim 25.

51. An absorbent article of claim 50, said article selected from the group consisting of a disposable diaper, a disposable incontinence pad and a disposable feminine hygiene pad.

52. An absorbent article, said article comprising a topsheet, an intermediate layer, an absorbent layer, and a backsheet, wherein the intermediate layer is the layer next to the topsheet, wherein the topside surface of said intermediate layer is the surface in contact with the topsheet, and wherein the intermediate layer is a composition of claim 1, and wherein said topside surface comprises the anionic surfactant and the polymeric biguanide.

53. An absorbent article of claim 52, said article selected from the group consisting of a disposable diaper, a disposable incontinence pad and a disposable feminine hygiene pad.

54. An absorbent article, said article comprising a topsheet, an intermediate layer an absorbent layer, and a backsheet, wherein the intermediate layer is the layer next to the topsheet, wherein the topside surface of said intermediate layer is the surface in contact with the topsheet, and wherein the intermediate layer is a composition of claim 25, and wherein said topside surface is comprises the polymeric biguanide.

55. An absorbent article of claim 54, said article selected from the group consisting of a disposable diaper, a disposable incontinence pad and a disposable feminine hygiene pad.
